(19) United States
(12) Patent Application Publication
(10) Pub. No.: US 2019/0117917 A1
(43) Pub. Date: Apr. 25, 2019

(54) TWO-PART PLASTIC BLANK SET

(71) Applicant: Claudia MATTERN, Emmetten (CH)

(72) Inventor: Claudia MATTERN, Emmetten (CH)

(21) Appl. No.: 16/092,043

(22) PCT Filed: Mar. 30, 2017
(86) PCT No.: PCT/EP2017/057515

§ 371 Date: Oct. 8, 2018

(30) Foreign Application Priority Data
May 25, 2016 (EP) 16171312.8

(51) Int. Cl. A61M 15/08; A61M 31/00; B65D 1/09; A61M 15/00

(52) U.S. Cl. CPC A61M 15/08; A61M 31/00; A61M 11/02; A61M 15/0031; A61M 2207/00; B65D 1/095

(57) ABSTRACT

The present invention relates to a two-part plastic blank set for the production of a drug applicator for storing a unit dose of a drug preparation and for administering that unit dose into the human nose.

### TECHNICAL FIELD

The present invention relates to a two-part plastic blank set, preferably for the production of a drug applicator for storing a unit dose of a drug formulation and for administering that unit dose into the human nose; and a drug applicator that can be produced therefrom.

### BACKGROUND

In some drug preparations or drug formulations for the human or animal body, precise dosages are crucial. In order to ensure such a precise dosage by the user, dosage units are provided which contain the intended amount of the drug preparation and which, in use, deliver precisely that intended amount to the human or animal body. In addition, drug applicators are also known which are intended to be used only once and perform a dual function: firstly, they contain a predetermined dosage amount of the drug preparation. Secondly, they serve not only to provide the drug preparation, but also to deliver it to the desired location in the desired amount. Drug applicators of this kind, which can have a small volume on the order of magnitude of one cubic centimetre or less, generally have the shape of a small vial and are squeezed at the sides to deliver the content of the container by pressing out the content of the container. Once used, the emptied unit is disposed of. A drug applicator of this kind for the provision and administration of a unit dose in the nose is known from the EU Registered Design 001790908-0001 or DE 10 2010 033 015 A1.

That drug applicator has a bottom part and a lid part, between which is formed a receiving space for a drug formulation. The bottom part and the lid part each have a peripheral sealing rim for joining and sealing the lid part to the bottom part. The bottom part is provided with an applicator tube, which terminates in the receiving space through an inner mouth. Relative to the plane of the joined sealing rims, the applicator tube provided for insertion into the nose projects beyond the sealing rims at a shallow angle, beginning at the receiving space, and in this way covers a supporting surface portion of the sealing rim on the bottom part. The bottom part and lid part are formed integrally via a film hinge.

A one-part plastic blank to form such a drug applicator can be manufactured inexpensively as an injection-moulded part under mass production conditions. It is, however, difficult to fill and seal that plastic blank, because of the geometrical arrangement of the applicator tube. For the filling process, precise positioning of the blank is necessary while it is still open, at which time the applicator tube is in the way. For the subsequent sealing procedure, when the lid part is sealed onto the bottom part with the applicator tube, it is necessary to apply counter-pressure on the sealing rim of the bottom part, which extends over the entire sealing surface. Only in this way can an all-round, hermetic seal be ensured. In the region of the applicator tube, however, the sealing rim is covered by the latter, so that access to the sealing rim here, in order to apply the counter-pressure, is prevented.

In technical implementation, it has become apparent that an integral configuration of the lid part and bottom part is disadvantageous, since the industrial production and filling of an applicator formed from an integral workpiece is not possible in practice, and especially that there are difficulties in correctly placing the one-piece plastic blank in a filling and sealing apparatus. In particular, the integral workpiece cannot be filled and sealed fully automatically. One particular difficulty in filling and sealing an integral workpiece is that the bottom part of the workpiece has to be placed in exactly the right position in a holding means of a filling and sealing apparatus and the lid part joined to the bottom part via the film hinge must be kept at a predetermined and fixed angle relative to the bottom part in order to allow the bottom part to be filled and then sealed.

In addition, it has become apparent that an attachment member between the applicator tube and the seal is frequently bent during transport, as a result of which leaks can occur.

Furthermore, the drug applicators known in the state of the art only permit the delivery of drug formulations of very low viscosity, such as viscosities of no more than 1,000 mPa·s. It is also frequently the case in the state of the art that satisfactory dosing consistency is not achieved when, for example, monodose drug applicators are used daily, which are always supposed to administer a constant dose.

### SUMMARY

It is therefore an object of the present invention to provide a plastic blank set for the production of a drug applicator, preferably for storing a unit dose of a drug formulation and for administering that unit dose into the human nose, which overcomes the disadvantages of the state of the art, which in particular allows simple and rapid filling and sealing in a corresponding filling and sealing means and which avoids leaks during transportation of a drug applicator manufactured from it. It is also the intention to provide a drug applicator with which drug formulations of greater viscosity can be administered securely, especially achieving good dosing consistency.

This object is achieved by a two-part plastic blank set, preferably for the production of a drug applicator for storing a unit dose of a drug formulation and for administering that unit dose into the human nose, the plastic blank set comprising a bottom part and a lid part separate from the bottom part, the bottom part having a receiving space configuration enclosed by a sealing rim and wherein an applicator tube integrally moulded on extends from the receiving space configuration and is sealed at its free end by a seal integrally moulded on via an attachment member, the attachment member having a wall thickness of 0.1-0.5 mm, preferably 0.2-0.3 mm, and the lid part being of such a size as to completely cover the receiving space configuration of the bottom part when placed thereon, and having a sealing rim which can be sealingly joined to the sealing rim of the bottom part, wherein an attachment tab extending from the respective sealing rim, preferably in the same plane as the sealing rim, is moulded onto at least the lid part or the bottom part.

The attachment tab extending from one sealing rim or both sealing rims is especially helpful if product information is to be applied to the lid part or the bottom part. This can be done by for example attaching a label to the attachment tab or printing on the attachment tab. When a label is used, adhesive is prevented from entering the receiving space configuration. If both the sealing rim of the bottom part and the sealing rim of the top part have attachment tabs moulded on to them such that they face each other in the finished drug applicator, they can likewise be sealed together, which improves the tightness of the seal and the stability of the drug applicator manufactured. It has also been found that the attachment tab can be used particularly advantageously so that the bottom part and/or the top part can be placed in a holding means of a filling and sealing apparatus. It is only thanks to the presence of such an attachment tab that the industrial production of the drug applicator with the two-part plastic blank set becomes possible and allows the manufacture of more than 150,000 items per day with one machine, whereas when a one-part plastic blank is used, in which the top and bottom parts are joined together via a film hinge, only about 5,000 items per day can be manufactured with a filling and sealing apparatus which is otherwise similar in configuration.

In one embodiment, it may be contemplated that at least the sealing rim of the lid part or the sealing rim of the bottom part is formed with grooves on a surface opposite the bottom part or the top part.

The provision of grooves on the sealing rims of the lid part and/or bottom part makes it possible to achieve ultrasonic welding results with process security. Grooves as means of directing energy in a specific direction are known in the field of ultrasonic welding and in particular have the task of rapidly inducing the plasticising of the joining surfaces by concentrating energy. When a means of directing energy is used, a seam is formed at the welding site, which improves the strength and uniformity of the welding result. In this connection, at least one sealing rim is provided with small grooves on the surface opposite the other sealing rim.

In addition, it may be contemplated that the receiving space configuration is shaped like a spherical cap or spherical segment. The receiving space configuration can, however, also have an oval outline, for example, in which case the peripheral sealing rim and the sealing rim of the lid part are then matchingly oval in shape.

It may also be contemplated that the lid is substantially flat in configuration.

One embodiment is characterised by the fact that the plastic blank set is made from polyolefin, such as polypropylene or polyethylene.

In a further embodiment, it may be contemplated that the polyolefin contains an inorganic additive.

In a still further embodiment, it is proposed that the inorganic additive is a pigment, preferably titanium dioxide (TiO₂) surface-treated titanium dioxide or a mixture of the two.

When an inorganic additive is used in the material of the bottom part, the top part and the applicator tube, it has been found that this can reduce physical/chemical interactions between the drug applicator and the drug formulation contained therein, especially adsorption of the formulation to the plastic material. The additive is preferably present in the plastic material in an amount between 0.1 and 10% by weight. The plastic material can preferably be processed by means of extrusion injection moulding.

The invention also relates to a drug applicator which can be manufactured by welding, preferably ultrasonically welding, the bottom part to the lid part of the two-part plastic blank set in accordance with one of claims 1 to 7 via the respective sealing rims and which is filled with a drug formulation, the drug formulation preferably having a viscosity of more than 2,500 mPa·s, preferably more than 3,000 mPa·s, even more preferably a viscosity in a range from 3,000-8,000 mPa·s, as measured with a Brookfield HBDV-3 viscometer, spindle CP41, at a temperature of 20° C. The Brookfield HBDV-3 device includes a cone-plate system.

The viscosity of the drug formulation is determined in accordance with the European Pharmacopoeia 9.1, Chapter 2.2.10. In one embodiment, the drug applicator is intended for use in the administration of the drug formulation contained in the drug applicator into the nose of a mammal, preferably a human.

It may also be contemplated that the administration is intended for the therapeutic or prophylactic treatment of the mammal.

A further embodiment is characterised by the fact that, after being dispensed from the drug applicator, the drug formulation forms a protective film against substances in the nose which are harmful to health, preferably against nanoparticles present in the atmosphere.

It goes without saying that other drug formulations can also be administered via the drug applicator of the invention, especially if they have a higher viscosity. With the drug applicator of the invention, the distribution of centrally active molecules can be optimised for the benefit of the central nervous system. In addition, it is possible to modify the release of the drug.

Finally, it may be contemplated that the bottom part, the lid part and the applicator tube have a wall thickness of at least 0.1 mm, preferably 0.2 mm, even more preferably 0.2-2.0 mm, still more preferably 0.2-1.5 mm or yet more preferably 0.2-1.0 mm.

The applicator tube moulded onto the receiving space configuration is open towards the receiving space configuration. The free end of the applicator tube is, however, closed by a seal, preferably a detachable plug or pin. The seal is joined to the applicator tube via an attachment member, preferably a tear-off seam. In order for the content of the drug applicator to be delivered, the seal is removed from the applicator tube, e.g. by twisting it about its longitudinal axis. The attachment member is preferably located in the interior of the applicator tube, so that there is no remaining burr projecting from the applicator tube in use, so that it cannot cause any injury when inserted into a user's nose for example.

The drug applicator produced in accordance with the invention is characterised in particular by a receiving space formed by the lid part and the bottom part which has a wall bulging outwards and an opposing wall provided with a peripheral ridge which engages in the edge of the cavity of the convex wall, the convex wall being formed in such a way that it can be pressed smoothly onto the ridge and the region of the opposing wall located therebetween.

When pressed together, the convex wall abuts the ridge and the opposing wall substantially without any space remaining between them, so that virtually the entire content is squeezed out of the receiving space, without any residual amount worth mentioning being able to remain, because the convex wall rests smoothly against the opposing wall, around the ridge, without forming any creases. The ridge, which preferably has a substantially conical shape in cross-section, with a rounded tip and slightly rounded sides, fills out the space that inevitably forms when a convex wall is pressed against its outer edge. Since that space is filled out by the ridge, no residual amount of the container content can remain there.

Suitable materials for the lid part and the bottom part are, for example, polyolefins, such as polypropylene, polyethylene or copolymers of ethylene and propylene, without the invention's being limited to these.

It has surprisingly been found in accordance with the invention that by providing a two-part plastic blank set, production engineering difficulties in filling and sealing a drug applicator made therefrom can be avoided. It is only by providing this two-part plastic blank set that the industrial production of drug applicators is possible at a production speed of, for example, at least 200 drug applicators per minute. That is about 10 times as many as has so far been possible when filling a one-part applicator. Filling the drug applicator of the invention fully automatically enables more accurate filling by dose and 100% ultrasonic welding. The drug applicator of the invention makes it possible for the first time to deliver a drug formulation of greater viscosity, i.e. more than 3,500 mPa·s, into the nose of a mammal. The drug applicator of the invention also allows a dosing consistency of about ±3% when drug applicators filled with identical drug formulation are used successively as monodose applicators over a lengthy period.

Furthermore, it has also surprisingly been found that by setting the wall thickness of the attachment member between the applicator tube and the seal appropriately, the transport safety of the drug applicators produced can be improved. In particular, the attachment member is prevented from bending, which avoids leaks.

### BRIEF DESCRIPTION OF THE DRAWINGS

Further details of the invention will become clear from the following description and from the enclosed drawings. There,

FIG. 1 shows a perspective view of a two-part plastic blank set comprising a bottom part and a top part in accordance with the invention; and

FIG. 2 shows a drug applicator produced from a lid part and a bottom part in accordance with the invention in a perspective view.

### DETAILED DESCRIPTION

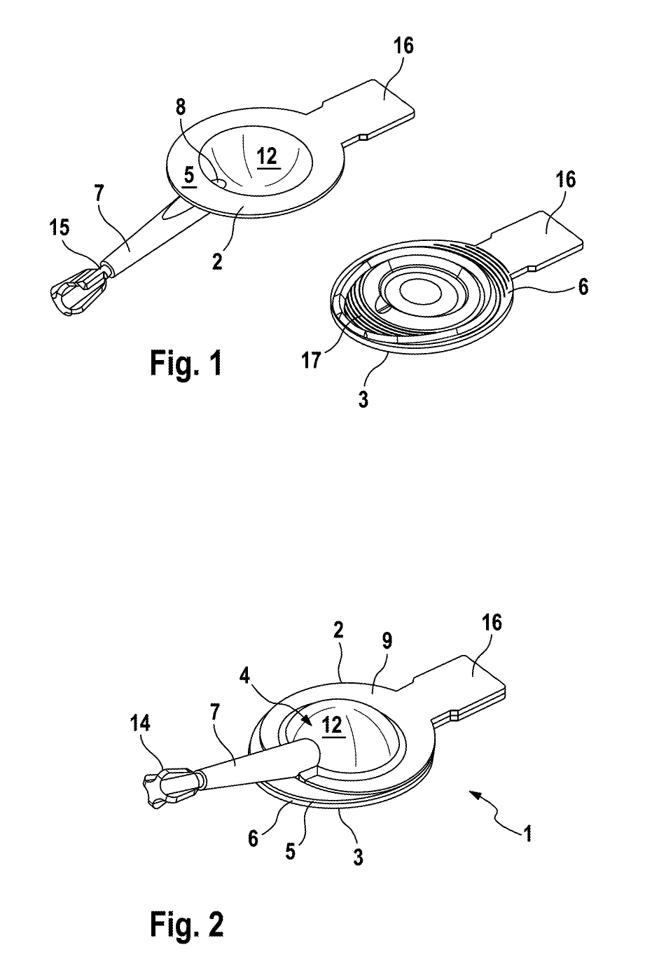

FIG. 1 shows a perspective view of a two-part plastic blank set for the production of a drug applicator. The plastic blank set comprises a bottom part 2 and a lid part 3. The bottom part 2 has a receiving space configuration 12 which is enclosed by a sealing rim 5 and which is configured substantially in the shape of a spherical cup. Starting from the receiving space configuration 12, there extends an applicator tube 7 which is moulded on integrally, and which is sealed at its free end by a seal 14 likewise moulded on integrally via an attachment member 15. The size of the lid part 3 is such that it can completely cover the receiving space configuration 12 of the bottom part 2. The lid part 3 also has a peripheral sealing rim 6 which can be sealingly joined to the sealing rim 5 of the bottom part 2. The lid part 3 is configured to be substantially flat and has a sealing rim 6 with grooves 17 as means of directing energy in a specific direction.

Formed in the receiving space configuration 12 is a mouth 8, by means of which the applicator tube 7 leads into the receiving space configuration 12.

Both the bottom part 2 and the lid part 3 have attachment tabs 16, which extend in the same plane from the respective sealing rim. These attachment tabs may be printed on on their sides facing away from the other part, or may be provided with an adhesive label. In order to manufacture a drug applicator of the invention 1, as shown in FIG. 2, from the two-part plastic blank set of the invention, the bottom part 2 is filled with a drug in a suitable filling and sealing apparatus and then sealed with the lid part 3.

For this purpose, the bottom part can be placed in a filling and sealing apparatus, such as is known from EP 2 626 304 B1 for example. Unlike the procedure described there, using a one-part blank, the bottom part 2 is first inserted and filled in accordance with the present invention, and after the bottom part 2 has been filled, the lid part 3 is placed on the bottom part 2 in such a way that its sealing rim 6 comes to rest on the peripheral sealing rim 5 of the bottom part 2 running round the receiving space configuration 12. The presence of an attachment tab on the bottom part or top part ensures that it can be placed securely in a mount of the filling and sealing apparatus. The welding, preferably ultrasonic welding, is then carried out using routine methods known in the state of the art.

A drug applicator 1 produced in this way is shown in FIG. 2. The bottom part 2 and top part 3 together form a receiving space 4, in which a drug formulation is received.

For use, the seal 14 is first of all broken off, or twisted off, and removed via the attachment member 15. After that, the applicator tube 7 is introduced into a nostril, whereupon squeezing the receiving space configuration 12 with the finger and thumb forces the drug formulation stored in the receiving space 4 through the applicator tube 7 and into the nose. The attachment member 15 has a wall thickness in the range of 0.1-0.5 mm, preferably 0.2-0.3 mm.

The features of the invention disclosed in the above description and the claims can be essential both individually and in any combination to implementing the invention in its various embodiments.

1. A two-part plastic blank set, the plastic blank set comprising a bottom part and a lid part separate from the bottom part, the bottom part having a receiving space configuration enclosed by a sealing rim, and wherein an applicator tube integrally moulded on extends from the receiving space configuration and is sealed at its free end by a seal integrally moulded on via an attachment member, the attachment member having a wall thickness of 0.1-0.5 mm, and the lid part being of such a size as to completely cover the receiving space configuration of the bottom part when placed thereon, and having a sealing rim which can be sealingly joined to the sealing rim of the bottom part, wherein an attachment tab extending from the respective sealing rim, is moulded onto at least the lid part or the bottom part.

2. The two-part plastic blank set as claimed in claim 1, wherein at least the sealing rim of the lid part or the sealing rim of the bottom part is formed with grooves on a surface opposite the bottom part or the top part.

3. Two-part plastic blank set as claimed in claim 1, wherein the receiving space configuration has a shape of a spherical cap or spherical segment.

4. Two-part plastic blank set as claimed in claim 1, wherein the lid part is substantially flat in configuration.

5. Two-part plastic blank set as claimed in claim 1, wherein the plastic blank set is made from polyolefin.

6. The two-part plastic blank set as claimed in claim 5, wherein the polyolefin contains an inorganic additive.

7. The two-part plastic blank set as claimed in claim 6, wherein the inorganic additive is a pigment, preferably titanium dioxide (TiO₂) surface-treated titanium dioxide or a mixture of the two.

8. A drug applicator which is manufactured by welding, the bottom part to the lid part of the the two-part plastic blank set as claimed in claim 1 via the respective sealing rims and which is filled with a drug formulation.

9. The drug applicator as claimed in claim 8 for use in the administration of the drug formulation contained in the drug applicator into the nose of a mammal.

10. The drug applicator as claimed in claim 9, wherein the administration is intended for the therapeutic or prophylactic treatment of the mammal.

11. The drug applicator as claimed in claim 8, wherein, after being dispensed from the drug applicator, the drug formulation forms a protective film against substances in the nose which are harmful to health.

12. The two-part plastic blank set as claimed in claim 1, wherein the bottom part, the lid part and the applicator tube have a wall thickness of at least 0.1 mm.
